(19) United States
(12) Patent Application Publication
(10) Pub. No.: US 2026/0085071 A1
(43) Pub. Date: Mar. 26, 2026

(54) NOVEL CRYSTALLINE FORM OF 1-(8-BROMOPYRIDO[2,3- E][1,2,4]TRIAZOLO[4,3-A]PYRAZIN-4-YL)-NMETHYLAZETIDIN- 3-AMINE

(71) Applicant: JW PHARMACEUTICAL CORPORATION, Gyeonggi-do (KR)

(72) Inventors: Suha PARK, Gyeonggi-do (KR); Seiho CHIN, Gyeonggi-do (KR); Sumin LEE, Gyeonggi-do (KR)

(21) Appl. No.: 19/110,267

(22) PCT Filed: Sep. 29, 2023
(86) PCT No.: PCT/IB2023/059788

§ 371 Date: Mar. 10, 2025

(30) Foreign Application Priority Data
Sep. 29, 2022 (EP) 22198685.4

(51) Int. Cl. C07D 471/14; A61K 31/4985

(52) U.S. Cl. CPC C07D 471/14; A61K 31/4985

(57) ABSTRACT

Novel crystalline forms of 1-(8-bromopyrido[2,3-e][1,2,4]triazolo[4,3-a]pyrazin-4-yl)-N-methylazetidin-3-amine is provided along with pharmaceutical compositions comprising the same. Also disclosed is the use of the novel polymorph for the treatment of diseases, such as atopic dermatitis (AD), itch, pruritus and various forms of urticaria for example chronic idiopathic urticaria subtypes.

### SUMMARY OF THE INVENTION

The present invention relates to a novel crystalline form of 1-(8-bromopyrido[2,3-e][1,2,4]triazolo[4,3-a]pyrazin-4-yl)-N-methylazetidin-3-amine, pharmaceutical compositions comprising the novel crystalline form of 1-(8-bromopyrido[2,3-e][1,2,4]triazolo[4,3-a]pyrazin-4-yl)-N-methylazetidin-3-amine and to the use of the novel crystalline form for the treatment of diseases, such as atopic dermatitis (AD), itch, pruritus and various forms of urticaria for example chronic idiopathic urticaria subtypes, such as cholinergic urticaria. Also provided herein is a method of preparing the crystalline form of the invention.

### BACKGROUND OF THE INVENTION

U.S. Pat. No. 9,586,959 relates, among other compounds, to the compound 1-(8-bromopyrido[2,3-e][1,2,4]triazolo[4,3-a]pyrazin-4-yl)-N-methylazetidin-3-amine and pharmaceutically acceptable salts thereof as well as pharmaceutical compositions comprising the same. The patent discloses the preparation of a number of salts of the compound 1-(8-bromopyrido[2,3-e][1,2,4]triazolo[4,3-a]pyrazin-4-yl)-N-methylazetidin-3-amine.

1-(8-bromopyrido[2,3-e][1,2,4]triazolo[4,3-a]pyrazin-4-yl)-N-methylazetidin-3-amine exhibits a strong histamine 4 receptor inhibitory effect and shows suppressive effects against histamine induced infiltration of inflammatory cells such as mast cells and eosinophils. The compound thus has strong anti-inflammatory and anti-itching effects and may therefore be useful for treating a range of diseases such as those disclosed in U.S. Pat. No. 9,586,959, including AD.

Different crystalline solid forms of chemical compounds may have distinct physical properties such as e.g. chemical stability, physical stability, hygroscopicity, melting point, solubility, dissolution rate, morphology and bioavailability which make them more or less suitable as the selected active ingredient in a pharmaceutical product.

In addition, a chemical entity may exist in several different crystalline solid forms and these include different polymorphic forms that share the same sum formula (e.g. anhydrates) and different solvates (e.g. hemihydrate, monohydrate and dihydrates) of the same chemical entity which do not share the same sum formula. Such crystalline solid forms have distinct crystal structures and vary in physical properties as mentioned above. The different crystalline solid forms can be distinguished from each other by e.g. melting point, XRPD pattern, spectral characteristics (e.g. FT-IR, Raman and ¹³C CP/MAS-NMR), and other physical and chemical properties. Chemical entities can also exist in amorphous form.

The actual crystalline form selected therefore plays an important role in the development and manufacture of an active pharmaceutical ingredient. Should a single crystal form be required, it is important that the crystallization process be robust and reliably produce the desired crystalline form in polymorphically pure form and that the crystalline form does not change (e.g. interconvert to a different crystalline form) during the relevant manufacturing processes, and/or during storage.

A number of different salts of 1-(8-bromopyrido[2,3-e][1,2,4]triazolo[4,3-a]pyrazin-4-yl)-N-methylazetidine have been identified. Some of the salts appear as anhydrate, others as monohydrates and dihydrates each in several polymorphic forms that interconvert upon drying or loses water at relatively low temperature and they are therefore not suitable for development as a pharmaceutical.

The novel crystalline form according to the invention is a crystalline form of the free base of 1-(8-bromopyrido[2,3-e][1,2,4]triazolo[4,3-a]pyrazin-4-yl)-N-methylazetidine-3-amine. The new crystalline form is hereinafter called from A.

Form A is thermodynamically stable up to 273° C. and is highly crystalline. Form A was also stable during stress testing at high temperature (60° C.) and high humidity (75%) for 4 weeks.

### SUMMARY OF THE INVENTION

The present invention relates to crystalline form A of 1-(8-bromopyrido[2,3-e][1,2,4]triazolo[4,3-a]pyrazin-4-yl)-N-methylazetidin-3-amine form A characterized by one or more XRPD reflections at approximately 2θ=7.0, 22.6 and/or 26.3 (±0.2 degrees).

The invention also relates to a pharmaceutical composition comprising the crystalline form mentioned above and a pharmaceutically acceptable carrier.

In one embodiment the invention relates to a compound or pharmaceutical composition as described above for the treatment of disease selected from atopic dermatitis, itch, pruritus, and various forms of urticaria, including chronic idiopathic urticaria subtypes.

### DETAILED DESCRIPTION OF THE INVENTION

The technical problem underlying the present invention is to circumvent the drawbacks of other crystalline and/or amorphous forms of 1-(8-bromopyrido[2,3-e][1,2,4]triazolo[4,3-a]pyrazin-4-yl)-N-methylazetidin-3-amine such as, ability to form crystals, filtration properties, solubility, thermodynamic properties, stability issues (e.g. due to water uptake), density, and transformation (e.g. interconversion to other polymorphic forms or hydrates/anhydrates) at varying degrees of humidity and during crystallization processes.

Hundred crystallization experiments resulted several polymorphic forms of the 1-(8-bromopyrido[2,3-e][1,2,4]triazolo[4,3-a]pyrazin-4-yl)-N-methylazetidin-3-amine.

Among these pure forms of A, E and F were obtained, but only form A were stable during gentle drying and attempts to prepare larger amounts of pure form E and F were not successful.

### Definitions

As used herein the term “rt” or “room temperature” indicates that the applied temperature is not critical and that no exact temperature value have to be kept. Usually, “rt” or “room temperature” is understood to mean temperatures of about 15° C. to about 25° C. [see e.g. EU Pharmacopoeia 7.5, 1.2 (2012)].

The term “solvate” as used herein describes a crystalline compound in which solvent molecules are incorporated into the crystal lattice of the compound in a stoichiometric or non-stoichiometric manner. If the solvent molecules are water the term “hydrate” is used herein.

The type of hydrate depends on the molar ratio of water molecules to 1-(8-bromopyrido[2,3-e][1,2,4]triazolo[4,3-a]pyrazin-4-yl)-N-methylazetidin-3-amine molecules.

The term “monohydrate” implies 0.8 to 1.2 mol water per mol of 1-(8-bromopyrido[2,3-e][1,2,4]triazolo[4,3-a]pyrazin-4-yl)-N-methylazetidin-3-amine.

An anhydrate is a crystal form which do not contain any water in the crystal lattice.

The term “non-hygroscopic” as used herein indicates that the increase in mass of a drug substance between about 0% to 80% relative humidity is less than 0.2% by weight.

In the context of the present invention, the term “XRPD reflection peak” denotes a particular 2Θ position in an XRPD pattern, wherein the signal-to-noise ratio (calculated according to item 2.2.46 of the European Pharmacopoeia) is greater than 3/1. “Absence of a peak” is herein defined as a peak having an intensity of at most 1%, such as 0.5% or 0.2%, of the highest peak in an XRPD of a sample of the compound of the invention, i.e no detectable XRPD peak above background signals.

In an XRPD pattern, the main characteristics of diffraction line profiles are 2Θ position, peak height, peak area and shape (characterized by, for example, peak width or asymmetry, analytical function, empirical representation). The 2Θ position is the most important factor as for example the intensity will be affected by sample preparation, and the width of the peaks by particle size. In addition to the diffraction peaks, an X-ray diffraction experiment also generates a more-or-less uniform background in an XRPD pattern, upon which the peaks are superimposed. Besides specimen preparation, other factors contribute to the background, for instance the sample holder, diffuse scattering from air and equipment, other instrumental parameters such as detector noise, general radiation from the X-ray tube, etc. The peak-to-background ratio can be increased by minimizing background and/or by choosing prolonged exposure times.

### Abbreviations

DSC: Differential Scanning Calorimetry
TGA: Thermogravimetric Analysis
XRPD: X-ray Powder Diffraction
¹³C CP/MAS NMR: ¹³C cross polarization magic angle spinning nuclear magnetic resonance
SXRD: Single crystal X-ray Diffraction

### BRIEF DESCRIPTION OF THE DRAWINGS

FIG. 1A: XRPD pattern for 1-(8-bromopyrido[2,3-e][1,2,4]triazolo[4,3-a]pyrazin-4-yl)-N-methylazetidin-3-amine form A (3-45° 2theta)

FIG. 1B: XRPD pattern for 1-(8-bromopyrido[2,3-e][1,2,4]triazolo[4,3-a]pyrazin-4-yl)-N-methylazetidin-3-amine form A (3-30° 2theta)

FIG. 2: DSC and TGA curve of 1-(8-bromopyrido[2,3-e][1,2,4]triazolo[4,3-a]pyrazin-4-yl)-N-methylazetidin-3-amine form A.

FIG. 3: The ORTREP drawing of the absolute crystal structure of crystalline form A of 1-(8-bromopyrido[2,3-e][1,2,4]triazolo[4,3-a]pyrazin-4-yl)-N-methylazetidin-3-amine form A.

FIG. 4: ¹³C CP/MAS NMR (14.1 T) spectrum of 1-(8-bromopyrido[2,3-e][1,2,4]triazolo[4,3-a]pyrazin-4-yl)-N-methylazetidin-3-amine, form A.

Results from Single Crystal Structure determination shown in Table 1.

TABLE 1
The crystal parameters from the single
crystal structure determination.

| Crystal form (Sample) | Form A |
|---|---|
| Empirical formula | C12 H12 Br N7 |
| Formula weight (g/mol) | 334.18 |
| Temperature/K | 120 |
| Crystal system | Monoclinic |
| Space group | P21/c |
| a/Å | 12.4941 (4) |
| b/Å | 8.3446 (2) |
| c/Å | 12.3436 (3) |
| α/° | 90 |
| β/° | 91.596 (3) |
| γ/° | 90 |
| Volume/Å³ | 1286.43 (6) |
| Z | 4 |
| Dx (Mg m⁻³) | 1.725 |
| μ/mm⁻¹ | 3.20 |
| F(000) | 671.0771 |
| Radiation | Mo Kα (λ = 0.71073) |
| θ values (°) | θmax = 32.2, θmin = 3.3 |
| Index ranges | h = −18→18, k = −12→12, |
| | l = −18→18 |
| No. of measured, independent and | 26471, 4307, 3433 |
| observed [I ≥ 2u(I)] reflections | |
| Refinement | |
| R[F2 > 2σ(F2)], wR(F2), S | 0.051, 0.134, 1.03 |
| No. of reflections | 4307 |
| No. of parameters | 182 |
| No. of restraints | 0 |
| No. of constraints | 20 |
| H-atom treatment | H-atom parameters constrained |
| (Δ/σ)max | <0.001 |
| Δρmax, Δρmin (e Å − 3) | 3.49, −1.63 |

Thus, in one embodiment the invention relates to the 1-(8-bromopyrido[2,3-e][1,2,4]triazolo[4,3-a]pyrazin-4-yl)-N-methylazetidin-3-amine form A.

In another embodiment the invention relates to crystalline 1-(8-bromopyrido[2,3-e][1,2,4]triazolo[4,3-a]pyrazin-4-yl)-N-methylazetidin-3-amine form A characterized by one or more XRPD reflections at approximately (°2θ) 7.0, 22.6 and/or 26.3 (±0.2 degrees).

In a further embodiment the invention relates to crystalline 1-(8-bromopyrido[2,3-e][1,2,4]triazolo[4,3-a]pyrazin-4-yl)-N-methylazetidin-3-amine from A characterized by one or more XRPD reflections at approximately (°2θ) 7.0, 14.3, 15.8, 22.6, 23.4 and/or 26.3 (±0.2 degrees).

In a further embodiment the invention relates to crystalline 1-(8-bromopyrido[2,3-e][1,2,4]triazolo[4,3-a]pyrazin-4-yl)-N-methylazetidin-3-amine form A characterized by one or more XRPD reflections at approximately (°2θ) 7.0, 22.6 and 26.3 (±0.2 degrees).

In a further embodiment the invention relates to crystalline 1-(8-bromopyrido[2,3-e][1,2,4]triazolo[4,3-a]pyrazin-4-yl)-N-methylazetidin-3-amine form A characterized by one or more XRPD reflections at approximately (°2θ) 7.0, 14.3, 15.8, 22.6, 23.4 and 26.3 (±0.2 degrees).

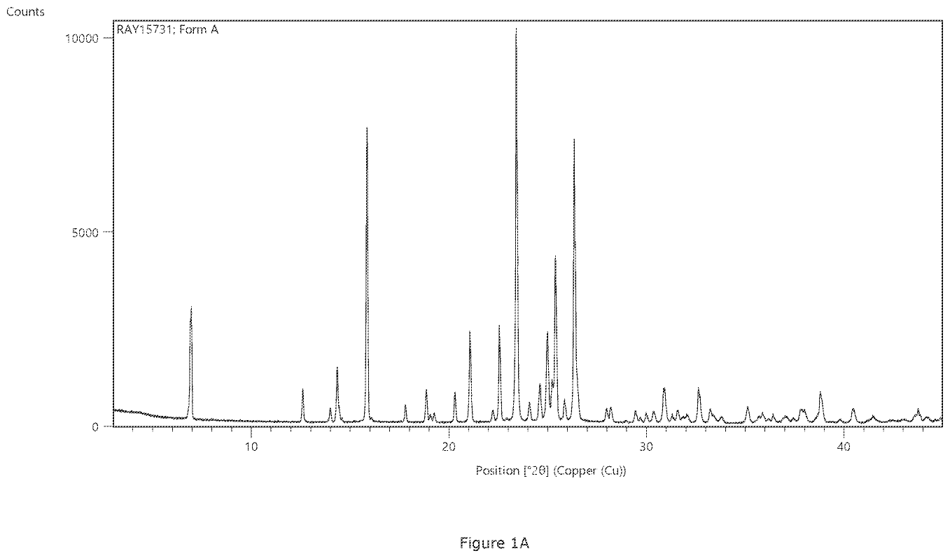

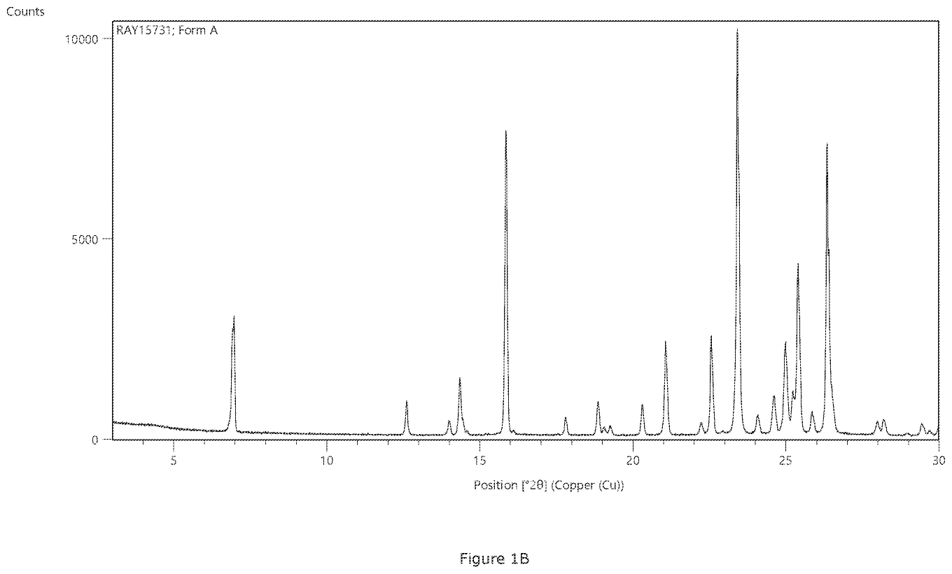

In a further embodiment the invention relates to crystalline 1-(8-bromopyrido[2,3-e][1,2,4]triazolo[4,3-a]pyrazin-4-yl)-N-methylazetidin-3-amine form A, wherein the crystalline compound has an XRPD pattern essentially similar to the XRPD pattern in FIGS. 1A and/or 1B.

In a further embodiment the invention relates to crystalline 1-(8-bromopyrido[2,3-e][1,2,4]triazolo[4,3-a]pyrazin-4-yl)-N-methylazetidin-3-amine form A, wherein the crystalline compound has an XRPD pattern according to the XRPD pattern in FIGS. 1A and/or 1B.

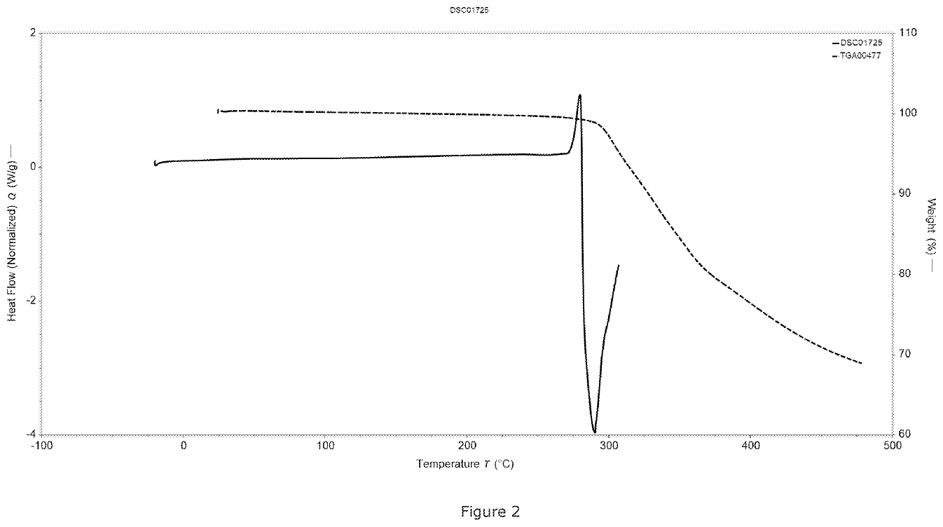

In one embodiment the crystalline 1-(8-bromopyrido[2,3-e][1,2,4]triazolo[4,3-a]pyrazin-4-yl)-N-methylazetidin-3-amine form A has an DSC and TGA curve essentially similar as shown in FIG. 2 consisting of a not separated endothermic-exothermic event with an onset at 273.6±2° C. and a corresponding weight loss in the TGA curve.

In a further embodiment the invention relates to crystalline 1-(8-bromopyrido[2,3-e][1,2,4]triazolo[4,3-a]pyrazin-4-yl)-N-methylazetidin-3-amine form A, wherein the crystalline compound is characterized by a solid state ¹³C CP/MAS NMR spectrum with peaks at one or more of 149.1, 148.1, 139.9, 128.0, 119.9, 112.7, 64.6, 63.1, 61.1, 53.6, 51.7, 37.7 and/or 35.4 ppm±0.2 ppm.

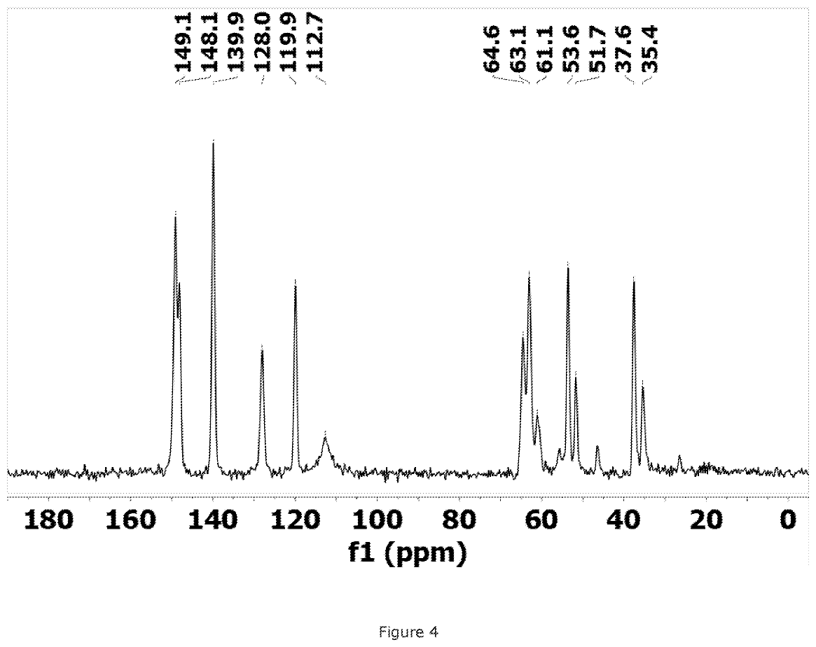

In a further embodiment the invention relates to crystalline 1-(8-bromopyrido[2,3-e][1,2,4]triazolo[4,3-a]pyrazin-4-yl)-N-methylazetidin-3-amine form A characterized by having a ¹³C CP/MAS NMR spectrum essentially similar to the ¹³C CP/MAS NMR spectrum in FIG. 4.

In a further embodiment the invention relates to crystalline 1-(8-bromopyrido[2,3-e][1,2,4]triazolo[4,3-a]pyrazin-4-yl)-N-methylazetidin-3-amine form A characterized by having a ¹³C CP/MAS NMR spectrum according to the ¹³C CP/MAS NMR spectrum in FIG. 4.

In a further embodiment the invention relates to crystalline 1-(8-bromopyrido[2,3-e][1,2,4]triazolo[4,3-a]pyrazin-4-yl)-N-methylazetidin-3-amine form A characterized by having the above ¹³C CP/MAS NMR spectrum and further characterized by one or more XRPD reflections at approximately (°2θ) 7.0, 22.6 and 26.3 and (±0.2 degrees).

In a further embodiment the invention relates to crystalline 1-(8-bromopyrido[2,3-e][1,2,4]triazolo[4,3-a]pyrazin-4-yl)-N-methylazetidin-3-amine form A wherein the crystalline compound is characterized by having the single-crystal X-Ray crystallography (SXRC) parameters as shown in table 1.

In a further embodiment of the present invention, 1-(8-bromopyrido[2,3-e][1,2,4]triazolo[4,3-a]pyrazin-4-yl)-N-methylazetidin-3-amine form A is characterized by having single crystal parameters which are substantially the same as those provided in Table 1.

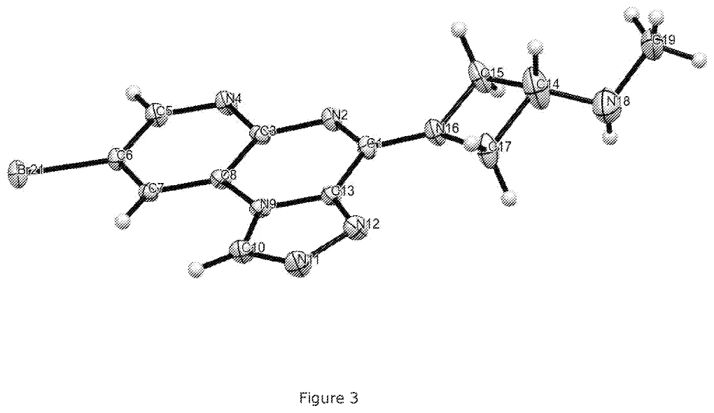

In a further embodiment of the present invention, 1-(8-bromopyrido[2,3-e][1,2,4]triazolo[4,3-a]pyrazin-4-yl)-N-methylazetidin-3-amine form A has a structure obtained by single-crystal X-Ray Diffraction (SXRD) as shown in FIG. 3.

In additional embodiments the invention relates to pharmaceutical composition comprising a crystalline compound as described above and a pharmaceutically acceptable carrier.

In an additional embodiment the invention relates to a pharmaceutical composition as above for use in the treatment of disease selected from atopic dermatitis, itch, pruritus, and various forms of urticaria.

In a particular embodiment the invention relates to a pharmaceutical composition as above wherein the forms of urticaria include chronic idiopathic urticaria subtypes, such as cholinergic urticaria.

The free base of 1-(8-bromopyrido[2,3-e][1,2,4]triazolo[4,3-a]pyrazin-4-yl)-N-methylazetidin-3-amine may be prepared as described in U.S. Pat. No. 9,586,959.

A further aspect of the present invention is directed to a pharmaceutical composition comprising the crystalline compound of the present invention and at least one pharmaceutically acceptable excipient. The pharmaceutical composition may be an oral dosage form, preferably a tablet and/or capsule.

In addition, the present invention relates to the use of the crystalline compound of the present invention for the preparation of a solid medicament.

In another embodiment the present invention relates to solid pharmaceutical compositions comprising an effective amount of the crystalline compound of the present invention and a pharmaceutically acceptable carrier as well as to processes of preparing the same. Moreover, the present invention is directed to the pharmaceutical composition of the present invention and/or the crystalline compound of the present invention for use in the treatment of any of the disease or disorders mentioned in U.S. Pat. No. 9,586,959, including diseases and disorders such as atopic dermatitis (AD), itch, pruritus and any of the various types of urticaria.

The pharmaceutical compositions of the present invention comprising the crystalline compound of the present invention may further comprise one or more pharmaceutically acceptable excipients. Such excipients are preferably selected from the group consisting of diluents, sweeteners, buffering agents, glidants, flowing agents, flavouring agents, lubricants, preservatives, surfactants, wetting agents, binders, disintegrants and thickeners. Other excipients known in the field of pharmaceutical compositions may also be used. Furthermore, the pharmaceutical composition may comprise a combination of two or more excipients also within one of the members of the above-mentioned group.

Suitable binders which can be used for the pharmaceutical compositions of the present invention comprising the crystalline compound of the present invention, further comprise e.g. alkylcelluloses such as methylcellulose, hydroxyalkylcelluloses such as hydroxymethylcellulose, hydroxyethylcellulose, hydroxypropylcellulose and hydroxybutylcellulose, hydroxyalkylalkylcelluloses such as hydroxyethylmethylcellulose and hydroxypropylmethylcellulose, carboxyalkylcelluoses such as carboxymethylcellulose, alkali metal salts of carboxyalkylcelluloses such as sodium carboxymethylcellulose, carboxyalkylalkylcelluloses such as carboxymethylethylcellulose, carboxyalkylcellulose esters, starches such as starch 1551, modified starches such as sodium carboxymethyl starch, pectins, chitin derivatives such as chitosan, heparin and heparinoids, polysaccharides such as alginic acid, alkali metal and ammonium salts thereof, carrageenans, galactomannans, tragacanth, agar-agar, gum arabic, guar gum and xanthan gum, polyacrylic acids and the salts thereof, polymethacrylic acids and the salts thereof, methacrylate copolymers, polyvinylalcohol, polyvinylpyrrolidone, copolymers of polyvinylpyrrolidone with vinyl acetate, polyalkylene oxides such as polyethylene oxide and polypropylene oxide and copolymers of ethylene oxide and propylene oxide, e.g. poloxamers and poloxamines, copovidone.

Suitable diluents which can be used for the pharmaceutical compositions of the present invention comprising the crystalline compound of the present invention further comprise e.g. calcium carbonate, dibasic calcium phosphate, dibasic calcium phosphate dihydrate, tribasic calcium phosphate, calcium sulphate, microcrystalline cellulose including silicified microcrystalline cellulose, powdered cellulose, dextrates, dextrin, dextrose excipient, fructose, kaolin, lactitol, lactose anhydrous, lactose monohydrate, mannitol, sorbitol, starch, modified starch, sodium chloride, sucrose, compressible sugar, confectioner's sugar, a spray-dried mixture of lactose monohydrate and microcrystalline cellulose (75:25), commercially available as Microcelac®, a co-processed spray-dried mixture of microcrystalline cellulose and colloidal silicon dioxide (98:2), commercially available as Prosolv®.

Suitable glidants which can be used for the pharmaceutical compositions of the present invention comprising the crystalline compound of the present invention further comprise e.g. talc, colloidal silicon dioxide, starch and magnesium stearate.

Suitable disintegrants which can be used for the pharmaceutical compositions of the present invention comprising the crystalline compound of the present invention further comprise e.g. starch, ion exchange resins, e.g. Amberlite, cross-linked polyvinylpyrrolidone, modified cellulose gum, e.g croscarmellose sodium, sodium starch glycolate, sodium carboxymethylcellulose, sodium dodecyl sulphate, modified corn starch, microcrystalline cellulose, magnesium aluminium silicate, alginic acid, alginate and powdered cellulose.

Suitable lubricants which can also be used for the pharmaceutical compositions of the present invention comprising the crystalline compound of the present invention further comprise e.g. magnesium stearate, calcium stearate, stearic acid, talc, polyethylene glycol, sodium lauryl sulphate and magnesium lauryl sulphate.

Some formulations, e.g. tablets may contain ingredients that have XRPD reflection peaks in the same position or area as the crystalline compound of the invention or have broad peaks. These may hide some of the XRPD pattern or peaks of the crystalline compound of the invention when the XRPD experiment is performed on a formulation comprising the crystalline compound of the invention as opposed to the pure crystalline salt alone. This means that one cannot always see all XRPD reflection peaks of the crystalline compound of the invention when an XRPD experiment is performed on a formulation of the crystalline compound

Thus, according to one embodiment, the invention relates to a pharmaceutical composition comprising a crystalline compound as defined herein together with pharmaceutically acceptable vehicle, excipient or pharmaceutically acceptable carrier(s), wherein said pharmaceutically acceptable vehicle, excipient or pharmaceutically acceptable carrier(s) comprises one or more ingredients which exhibit XRPD reflection peaks including one or more XRPD reflection peaks that overlap with and hide one or more XRPD reflection peaks of the crystalline compound of the invention.

The same issues may arise with ¹³C CP/MAS NMR where for example intense signals from a cellulose component should be expected in the spectral region 60-110 ppm and the peaks from stearate will be seen in the spectral region 15-40 ppm—along with a carbonyl peak around 172 ppm.

Thus, according to one embodiment, the invention relates to a pharmaceutical composition comprising a crystalline compound as defined herein together with pharmaceutically acceptable vehicle, excipient or pharmaceutically acceptable carrier(s), wherein said pharmaceutically acceptable vehicle, excipient or pharmaceutically acceptable carrier(s) comprises one or more ingredients which are characterized by a ¹³C CP/MAS NMR spectrum that may include one or more peaks that overlap with and hide one or more ¹³C CP/MAS NMR peaks of the crystalline compound of the invention.

The absence of other crystalline forms of 1-(8-bromopyrido[2,3-e][1,2,4]triazolo[4,3-a]pyrazin-4-yl)-N-methylazetidin-3-amine can be tested by comparing an XRPD pattern taken of any crystalline form of 1-(8-bromopyrido[2,3-e][1,2,4]triazolo[4,3-a]pyrazin-4-yl)-N-methylazetidin-3-amine or a salt thereof with an XRPD pattern of form F as obtained e.g. from example 1 and shown in FIG. 1A or 1B. For such a comparison, the XRPD pattern shown in FIGS. 1A and 1B can be taken as an XRPD pattern of 100% pure crystalline compound of form A or B of the present invention.

### Description of the Test Methods Used to Characterize the Polymorphic Forms Disclosed Herein

### X-Ray Powder Diffraction (XRPD)

XRPD patterns were collected with a PANalytical X'pert PRO MPD diffractometer using an incident Cu Kα radiation and operating at 45 kV and 40 mA. The XRPD patterns were collected in the 2 theta range from 3 to 45 degrees with a stepsize of 0.0066°, counting time of 148.93 s and in transmission geometry. In the incident beam path, an elliptically graded multilayer mirror together with a 4 mm fixed mask, fixed anti-scatter slit 1° and fixed divergence slits of ½° were placed to line focus the Cu Kα X-rays through the sample and onto the detector. At the diffracted beam path, a long antiscatter extension and antiscatter slits of 2 mm were placed to minimize the background generated by air. Furthermore, Soller slits of 0.02 rad were placed on both the incident and diffracted beam paths to minimize broadening from axial divergence.

The sample was placed on a 3 μm thick foil on a 96 high throughput well plate stage and oscillated in the X direction for better particle statistics. The diffraction patterns were collected using a PIXel RTMS detector with an active length of 3.347° and located 240 mm from the sample.

### Thermo Gravimetric Analysis (TGA):

TGA experiments were conducted using a TGA550 instrument from TA Instruments. About 1-10 mg of sample was loaded into a ceramic pan for the measurements. The sample temperature was ramped from 25 to 500° C. at 10° C./min. Nitrogen was used as the purge gas at a flow rate of 50 mL/min.

### Differential Scanning Calorimetry (DSC)

DSC: Heating rate of 10° C./min under a nitrogen atmosphere. About 1-2 mg of sample was loaded into an open aluminum pan for the measurements. Instrument Q20 from TA Instruments.

### Single-Crystal X-Ray Diffraction

Data were collected using a SuperNova, Dual diffractometer with an Atlas CCD area detector (Temperature: 120(2) K; Cu Kα Radiation λ=1.5418 Å; data collection method: ω scans).

Further details can be found in Table 1. Program(s) used to solve structure: CrysAlisPro, Agilent Technologies, Version 1.171.37.34 (release 22 May 2014 CrysAlis171 .NET), ShelXL (Sheldrick, 2008) used to refine structure and Olex2 (Dolomanov et al., 2009) for ORTEP drawings.

### Solid-State NMR Spectroscopy

¹³C CP/MAS NMR spectra were recorded at 298 K on a Bruker Avance III HD 600 NMR spectrometer (14.1 T) equipped with a 4 mm double-tuned (¹H-¹³C) CMP probe using a contact time of 6 ms, a recycle delay of 64 s, spin-rate of 14.1 kHz, 256 scans and high-power ¹H decoupling during the acquisition time of 45.9 ms. Prior to Fourier Transformation the time domain data (Free Induction Decay) was apodized by Lorentzian linebroadening of 5 Hz. All spectra are referenced to the chemical shift of the carbonyl group in α-glycine at 176.5 ppm (external sample).

The given error ranges in this application for the spectroscopic characteristics, including those in the claims, may be more or less depending on factors well known to a person skilled in the art of spectroscopy and may for example depend on sample preparation, such as particle size distribution, or if the crystal form is part of a formulation, on the composition of the formulation, as well as instrumental fluctuations, and other factors.

### EXAMPLES

### Example 1

11 mg of the free base of 1-(8-bromopyrido[2,3-e][1,2,4]triazolo[4,3-a]pyrazin-4-yl)-N-methylazetidin-3-amine was added to a test tube or 4 ml glass vial and 2 mL of CHCl₃:THF (1:3) mixture was added. The solution was heated with a heat gun until all solid material was visually dissolved. The solution was then filtered with a 0.45 μm syringe filter. The filtered solution was placed on the table in the fume cup with a loose lid for crystallization during slowly evaporation of the solvent system. The obtained crystals had a rhomboid shape. The XRPD pattern of form A is shown in FIGS. 1A and 1B.

1. Crystalline 1-(8-bromopyrido[2,3-e][1,2,4]triazolo[4,3-a]pyrazin-4-yl)-N-methylazetidin-3-amine form A characterized by one or more XRPD reflections at approximately (°2θ) 7.0, 22.6 and/or 26.3 (±0.2 degrees).

2. The crystalline compound according to claim 1, characterized by one or more XRPD reflections at approximately (°2θ) 7.0, 14.3, 15.8, 22.6, 23.4 and/or 26.3 (±0.2 degrees).

3. The crystalline compound according to claim 1, characterized by one or more XRPD reflections at approximately 7.0, 22.6 and 26.3 (±0.2 degrees).

4. The crystalline compound according to claim 1, characterized by one or more XRPD reflections at approximately (°2θ) 7.0, 14.3, 15.8, 22.6, 23.4 and 26.3 (±0.2 degrees).

5. The crystalline compound according to claim 1, wherein the crystalline compound has an XRPD pattern essentially similar to the XRPD pattern in FIG. 1A or 1B.

6. The crystalline compound according to claim 1, wherein the crystalline compound has an XRPD pattern according to the XRPD pattern in FIG. 1A or 1B.

7. The crystalline compound according to claim 1, wherein the crystalline compound is characterized by a solid state ¹³C CP/MAS NMR spectrum with peaks at one or more of 149.1, 148.1, 139.9, 128.0, 119.9, 112.7, 64.6, 63.1, 61.1, 53.6, 51.7, 37.7 and/or 35.4 ppm±0.2 ppm.

8. The crystalline compound according to claim 7 characterized by having a ¹³C CP/MAS NMR spectrum essentially similar to the ¹³C CP/MAS NMR spectrum in FIG. 4.

9. The crystalline compound according to claim 7 characterized by having a ¹³C CP/MAS NMR spectrum according to the ¹³C CP/MAS NMR spectrum in FIG. 4.

10. The crystalline compound according to claim 7, further characterized by one or more XRPD reflections at approximately (°2θ) 7.0, 22.6 and 26.3 (±0.2 degrees).

11. The crystalline compound according to claim 1 wherein the crystalline compound is characterized by having the single-crystal X-Ray crystallography (SXRC) parameters as shown in table 1.

12. The crystalline compound according to claim 1 wherein the crystalline compound is characterized by having the single-crystal X-Ray crystallography (SXRC) parameters which are substantially the same as those provided in Table 1.

13. The crystalline compound according to claim 1 wherein the crystalline has an DSC and TGA curve essentially similar as shown in FIG. 2 consisting of a not separated endothermic-exothermic event with an onset at 273.6±2° C. and a corresponding weight loss in the TGA curve.

14. A pharmaceutical composition comprising a crystalline salt according to claim 1 and a pharmaceutically acceptable carrier.

15. A method for the treatment of disease selected from atopic dermatitis, itch, pruritus, and various forms of urticaria, the method comprising:
administering to a subject in need thereof the crystalline compound of claim 1.

16. The method according to claim 15, wherein the disease is atopic dermatitis.
